(19) United States
(12) Patent Application Publication
(10) Pub. No.: US 2017/0120000 A1
(43) Pub. Date: May 4, 2017

(54) CENTRAL VENOUS CATHETER WITH REMOVABLE SIDE PORTS

(71) Applicant: Oscor Inc., Palm Harbor, FL (US)

(72) Inventors: Thomas P. Osypka, Palm Harbor, FL (US); NR Chandrasekar, Canton, MA (US); Jeff Drum, Tarpon Springs, FL (US); Michael J. Gelineau, Lutz, FL (US)

(73) Assignee: Oscor Inc., Palm Harbor, FL (US)

(21) Appl. No.: 15/339,140

(22) Filed: Oct. 31, 2016

Related U.S. Application Data
(60) Provisional application No. 62/248,724, filed on Oct. 30, 2015.

(51) Int. Cl. A61M 25/00; A61M 39/20; A61M 25/02

(52) U.S. Cl. CPC A61M 25/0097; A61M 25/0026; A61M 25/02; A61M 39/20; A61M 2025/0286

(57) ABSTRACT

A catheter assembly is disclosed that includes a primary hub having opposed proximal and distal end portions, and at least two secondary hubs operatively connected to the proximal end portion of the primary hub, each secondary hub including a body having opposed proximal and distal ends, the proximal end of each secondary hub body adapted and configured for connection with a removable side port.

### CROSS-REFERENCE TO RELATED APPLICATION

The subject invention claims the benefit of and priority to U.S. Provisional Patent Application Ser. No. 62/248,724 filed Oct. 30, 2015, the disclosure of which is herein incorporated by reference in its entirety.

### BACKGROUND OF THE INVENTION

1. Field of the Invention

The subject invention is directed to a medical device, and more particularly, to a central venous catheter with removable side ports each having an embedded hemostatic valve.

2. Description of Related Art

Central s catheters are typically used for medical procedures such as blood pressure monitoring, blood sampling and the administration of drugs and fluids to a patient. Such procedures often require that the catheter be left indwelling in the patient for an extended period of time.

Generally, a central venous catheter is surgically inserted into a major in, s the superior vena cava. Central venous catheters are typically introduced using percutaneous entry techniques, such as the well-known Seldinger technique. in the Seldinger technique, the physician makes an oblique entry into the vein with a beveled needle. A wire guide is then inserted through the bore of the needle about 5 to 10 cm into the vein. Thereafter, the needle is withdrawn, leaving the wire guide in place.

The catheter is then inserted over the wire guide, and advanced through the skin at the needle puncture site and into the vein. Once the catheter is in place within the vein, the wire guide is withdrawn. The Seldinger technique is a widely used procedure for introducing catheters and other interventional devices into the vasculature, and is normally carried out by the physician without complication.

Central venous catheters generally consist of a shaft having at least two or more lumens, extending from the proximal side to the distal side. With multi-lumen catheters, one lumen may be used for pressure monitoring, another lumen may be used for drug or fluid infusion, and still another lumen may be used for blood sampling. The central lumen can additionally serve as the wire guide lumen may be used during placement. The shaft material is commonly a softer grade Polyurethane and/or Tecothane.

As illustrated in FIG. 1, a prior art central venous catheter 10 features an elongated catheter shaft 12 having a soft atraumatic tapered tip 14 that is typically formed from a soft polymer, such as Polyurethane or a similar material, filled with contrast medium visible under X-ray. Such filling material may be BaSO₄ and or Tungsten of at least 20% or more, mixed into the polymer. The proximal side of the catheter 10 features a hub 16, to which is attached a plurality of side ports 18a through 18c, corresponding to each of the three lumens in catheter shaft 12. Each side port is normally 5 to 15 cm in length, connected directly through the proximal hub 16 towards the plurality of lumens of the catheter shaft 12.

The catheter shaft 12 is implanted into the patient's vein, typically the subclavian vein next to the patient's neck, with the proximal hub 16 and its side ports 18a-18c projecting out of the patient's vein. The proximal hub 16 normally has to be fixated by means of a suture or medical tape to the patient's neck. Due to the weight of the proximal huh 16 and the side ports 18a-18c, such an arrangement often creates discomfort for the patient, skin irritation and possibly infections. Also, due to the large size of the various side ports, the patient may we and or dislodge the catheter and/or by changing clothing.

Accordingly, it would be desirable to provide a central venous catheter that has a light weight proximal hub. It is further desired to provide a central venous catheter that has side ports which can be removed and that can be closed when not in use to additionally reduce the proximal catheter weight and maximize patient comfort.

### SUMMARY OF THE INVENTION

The subject invention is directed to a new and useful catheter assembly that includes includes a primary hub having opposed proximal and distal end portions, and at least two secondary hubs operatively connected to the proximal end portion of the primary hub, each secondary hub including an elongated body having opposed proximal and distal ends, the proximal end of each secondary hub body adapted and configured for connection with a removable side port. A multi-lumen catheter body extends from the distal end portion of the primary hub, and it includes at least two lumens.

The primary hub is adapted and configured to facilitate communication between the secondary hubs and the multi-lumen catheter. A flexible tube or conduit extends between the distal end of each secondary hub and the proximal end portion of the primary hub. An axial passage extends through each secondary hub between the proximal and distal ends thereof, and a hemostatic seal is arranged within the axial passage of each secondary hub.

The proximal end of each secondary hub body includes a leur-type connection for engagement with a fitting associated with a removable side port. Preferably, each secondary hub is color matched with a corresponding removable side port. The primary hub includes suturing structures for securing the primary hub to a patient's body.

The catheter assembly further includes a cap adapted and configured to plug exposed openings of the primary hub when the secondary hubs are removed. A locking system utilizing a key is configured selectively to lock the cap to the primary hub.

A fixation system is included and configured to secure the primary hub to a patient's body and prevent movement of the primary hub when not in use. The fixation system includes a bottom plate having at least two locking posts and a upper plate having holes for receiving the locking posts therein and wherein the primary hub is positioned fixed between the bottom plate and upper plate.

The subject invention is further directed to a central venous catheter assembly that includes a primary hub having opposed proximal and distal end portions, a plurality of secondary hubs operatively connected to the proximal end portion of the primary hub, each secondary hub including an elongated body having opposed proximal and distal ends, a multi-lumen catheter body extending longitudinally from the distal end portion of the primary hub, a plurality of removable side ports each adapted and configured for selective connection with the proximal end of a corresponding secondary hub body, and at least one cap adapted and configured to plug exposed openings of the primary hub when the side ports are removed.

In a preferred embodiment of the subject invention, at least three secondary hubs are operatively connected to the proximal end portion of the primary hub, and at least three removable side ports are operatively associated with the secondary hubs.

These and other features of the catheter assembly of the subject invention and the manner in which it is manufactured and employed will become more readily apparent to those having ordinary skill in the art from the following enabling description of the preferred embodiments of the subject invention taken in conjunction with the several drawings described below.

### BRIEF DESCRIPTION OF THE DRAWINGS

So that those skilled in the art to which the system of the subject invention appertains will readily understand how to make and use the subject invention without undue experimentation, preferred embodiments thereof will be described in detail herein below with reference to certain figures, wherein:

FIG. 1 is a perspective view of a prior art central venous catheter;

FIG. 2 is a perspective view of a proximal end of a central venous catheter constructed in accordance with a preferred embodiment of the subject invention, showing a plurality of removable side ports associated with a primary hub;

FIG. 2a is an enlarged detailed view of the central venous catheter in cross-section, showing a multi-lumen catheter body;

FIG. 3 is a perspective view of another embodiment of a primary hub constructed in accordance with the subject invention;

FIG. 4 is a perspective view of yet another embodiment of a primary hub constructed in accordance with the subject invention, showing a fixation system;

FIG. 5 is an exploded perspective view of the primary hub and fixation system of the catheter of FIG. 4;

FIG. 6 is a perspective view of still another embodiment of a primary hub constructed in accordance with the subject invention;

FIG. 7 is a top plan view of the primary hub of FIG. 6, showing a removable proximal end portion;

FIG. 8 is a perspective view of another embodiment of a primary hub and cap assembly having a locking feature;

FIG. 9 is an exploded perspective view of the primary hub and cap of FIG. 8; and

FIG. 10 is a detailed view in partial cross sectional illustrating the structure of the locking feature shown in FIGS. 8 and 9.

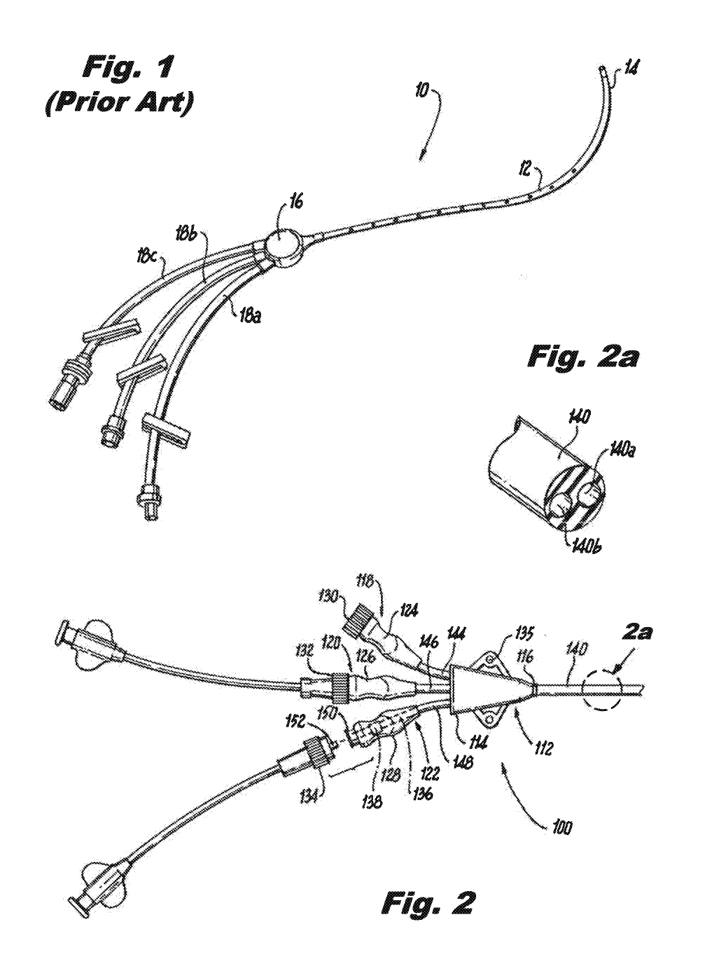

### DETAILED DESCRIPTION OF THE INVENTION

Referring now to the drawings wherein like reference numerals identify similar structural features of the claimed invention, there is illustrated in FIG. 2 a central venous catheter assembly in accordance with a preferred embodiment of the subject invention and designated generally by reference numeral 100. The catheter 100 includes a primary hub 112 having opposed proximal and distal end portions 114 and 116, and secondary hubs 118, 120 and 122 operatively connected to the proximal end portion 114 of the primary hub 112.

Each secondary hub 118, 120 and 122 includes an elongated body 124, 126 and 128 having opposed proximal and distal ends. The proximal end of each secondary hub body 124, 126 and 128 is adapted and configured for connection with a corresponding removable side port 130, 132 and 134. The primary hub 112 includes laterally opposed suturing structures 135 for securing the primary hub 112 to a patient's body.

A multi-lumen catheter body 140 extends from the distal end portion 116 of the primary hub 112, and it includes at least two lumens 140a, 140b (shown in FIG. 2a). At least one lumen extends longitudinally through the catheter body 140 to an exit port at a distal end thereof, and at least one lumen extends longitudinally through the catheter body 140 to a closed distal end portion thereof. The lumen that extends to the exit port can serve as the guidewire lumen for placement of the assembly 100.

The primary hub 112 is adapted and configured to facilitate communication between the secondary hubs 118, 120 and 122 and the multi-lumen catheter body 140. A plurality of flexible tubes 144, 146 and 148 extend between the distal end of each secondary hub 118, 120 and 122 and the proximal end portion 114 of the primary hub 112. The flexible tubes 144, 146 and 148 are inserted into openings (not shown) of the primary hub 112 which leads to the multi-lumen catheter body 140.

An axial passage 136 extends through each secondary hub body 124, 126, 128 between the proximal and distal ends thereof, and a hemostatic seal 138 (shown schematically) is arranged within the axial passage of each secondary hub 118, 120 and 122. The seal 138 is preferably molded out of a medical grade silicone with shore hardness softer than 30D, featuring a cut opening to allow the side port to engage and open the port when connected, but sealing the opening when disconnected and disengaged. A suitable seal is disclosed in commonly assigned U.S. Pat. No. 8,974,420, the disclosure of which is incorporated herein by reference in its entirety.

The proximal end of each secondary hub body 118, 120 and 122 includes a conventional leur-type connection 150 for engagement with a fitting 152 associated with each removable side port 130, 132 and 134. Preferably, each secondary hub 118, 120 and 122 is color matched with a corresponding removable side port 130, 132 and 134. The removable side ports can have different functions. For example, removable side port 130 may be used for blood sampling, whereas removable side ports 132 and 134, which have associated tubes 136 and 138, respectively, may be utilized for drug or fluid infusion, or pressure monitoring or the like.

The configuration of the subject invention dramatically reduces the overall hub size and weight and will benefit the patient comfort and prevent skin irritations, while preventing dirt or air from entering the side port lumens when they are not in use. In addition, removal of the side ports greatly reduces the likelihood of a patient dislodging the catheter and prevents injury. The catheter assembly 100 of the subject invention is not limited to a central venous catheter configuration as shown, as it is envisioned that this configuration featuring removable side ports may be employed as a peripherally inserted central catheters (PICC), an arterial catheters or other like catheter devices.

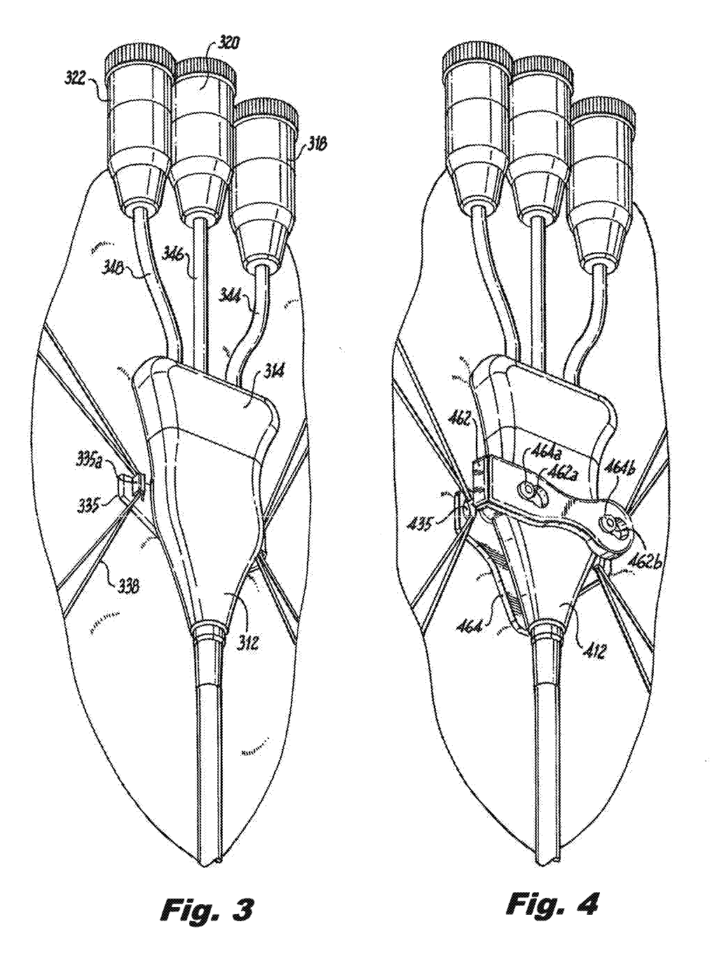

With reference to FIG. 3, another embodiment of a primary hub 312 is shown. Similar to primary hub 112, primary hub 312 is attached to removable side ports 318, 320 and 322 through flexible tubs 344, 346 and 348 at a proximal end 314. Primary hub 312 further includes laterally opposed suture tie downs 335. In this embodiment, suture tie downs 335 are outwardly extending channels 335a for threading sutures 338 therethrough.

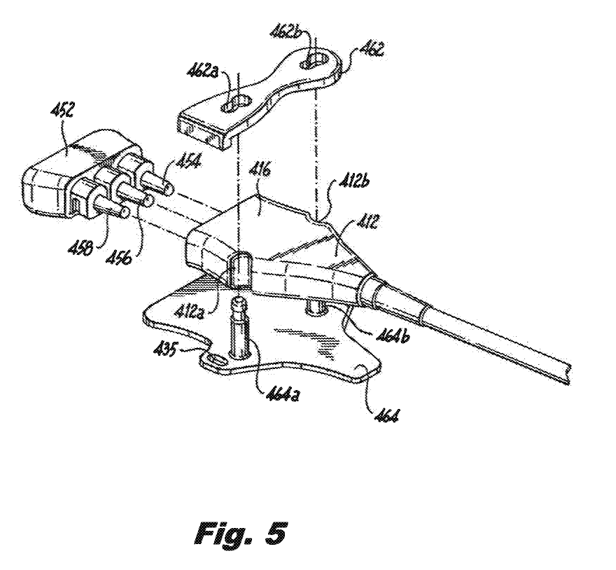

Referring to FIGS. 4 and 5, yet another embodiment is shown for the central venous catheter 400. This embodiment includes a fixation system to secure the catheter 400 to the skin entrance point of the patient. The fixation system will prevent the catheter from moving even if the patient is active thereby reducing discomfort and trauma to the entrance point. The fixation system includes an upper plate 462 and a bottom plate 464 configured to attach with and secure the primary hub 412. The bottom plate 464 includes suture tie down holes 435 for inserting sutures therein. Upwardly extending locking posts 464a, 464b are positioned adjacent each suture hole 435. The primary hub 412 includes laterally opposed semi-circular cut-outs 412a, 412b for slideably engaging with the locking posts 464a, 464b.

The upper plate includes a pair of slots 462a, 462b corresponding to the location of the locking posts 464a, 464b when aligned with the bottom plate 464. Each slot 462a, 462b has a larger circular hole for receiving the posts therein and an adjoined smaller hole for maintaining the posts 464a, 464b in position. It is envisioned to secure the fixation device to the patient, the bottom plate 464 will be sutured into position. The proximal hub 412 is slid on top of the bottom plate 464 with the posts 464a, 464b engaged within the semi-circular cut-outs 412a, 412b. Next, the upper plate 462 is lowered over the proximal hub 412, aligning the slots 462a, 462b with the posts 464a, 464b. Once each post 464a, 464b is positioned within the corresponding slot 462a, 462b, the upper plate 462 is shifted so that the posts 464a, 464b are locked within the smaller hole (shown best in FIG. 4).

FIG. 5 further illustrates a cap 452 that is used to plug the proximal hub 412 when the side ports 130, 132, 134 are removed. The ability to remove the side ports 130, 132, 134 when the catheter is not in use provides a light weight, comfortable catheter. The cap 452 includes individual plugs 454, 456, 458 that correspond to openings (shown in FIG. 9) of the primary hub 412. The cap 452 prevents air and/or dirt from entering the catheter 100 when not in use.

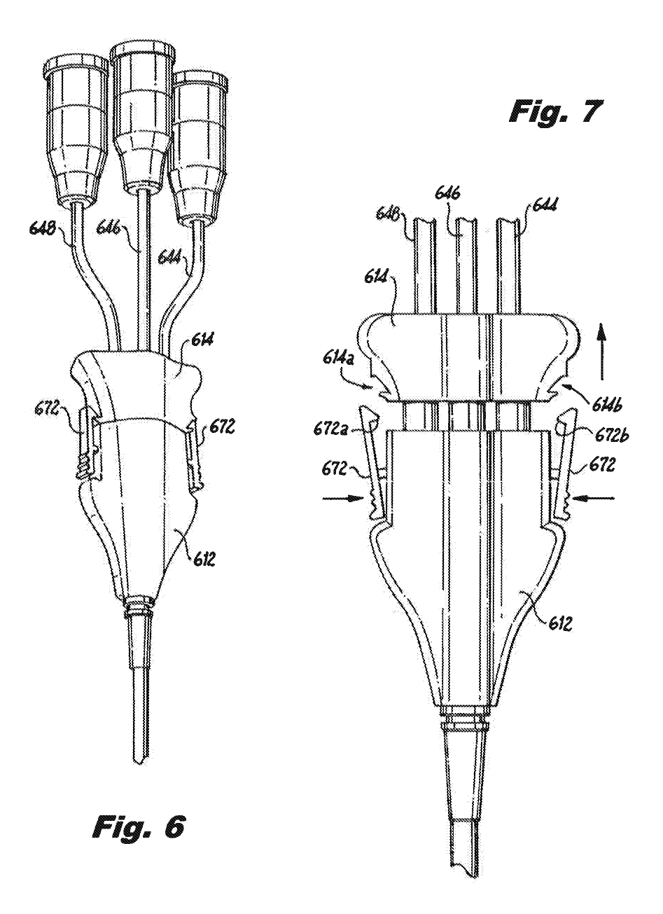

Referring to FIGS. 6 and 7, still another embodiment of the primary hub 612 is shown allowing for removal of the proximal end 614. Laterally opposed flexible tabs 672 are positioned along the proximal hub 672. Each flexible tab 672 includes a hook 672a, 672b which engages corresponding grooves 614a, 614b of the proximal end 614. As best seen in FIG. 7, a user pushes a portion of the flexible tabs 672 closest to the primary hub 612 causing the flexible tabs 672 to hinge outwardly. The proximal end 614 is pulled away from the proximal hub 612 exposing flexible tubes 644, 646, 648 thereby allowing removal of the flexible tubes 644, 646, 648 and side ports 130, 132 and 134 when the catheter 100 is not in use.

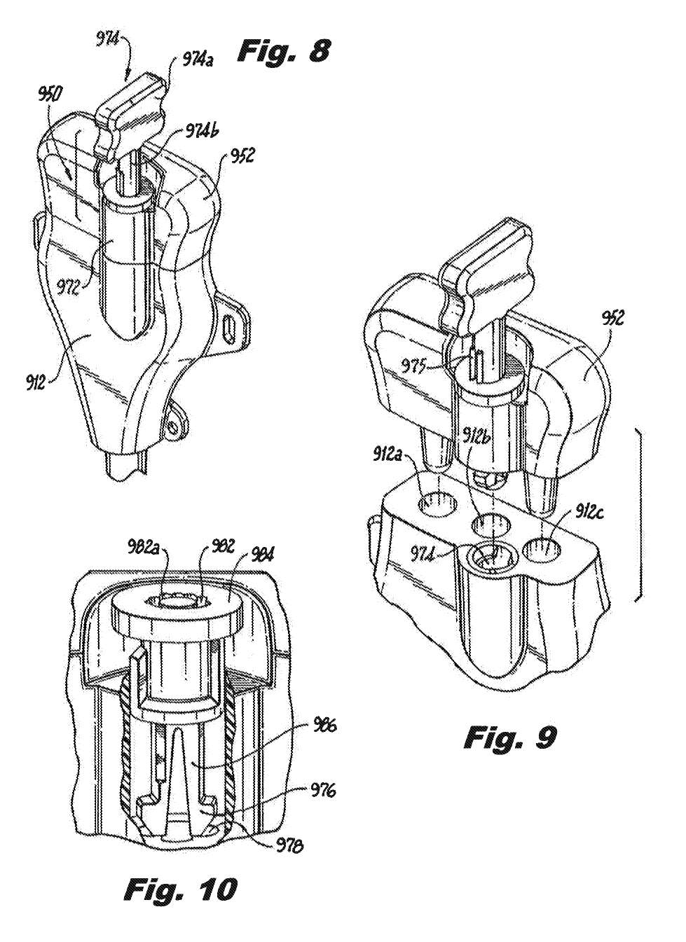

FIGS. 8-10 illustrate another embodiment of the primary hub 912 related to the central venous catheter 100 for safeguarding the openings 912a, 912b, 912c of the proximal hub 912 when the catheter is not in use. Recently it has been noted that patients may abuse the ports for injecting illegal and recreational drugs posing a great risk for patients and expense for hospitals. In this embodiment a locking feature 950 is integrated with the cap 952 to prevent usage of the openings 912a, 912b, 912c when not intended by a doctor or nurse. The locking feature 950 includes a key 974 (shown in FIGS. 8 and 9) to lock and unlock the cap 952 with the primary hub 912.

The key 974 includes a bow 974a and a longitudinally extending blade 974b. The blade 974a has a plurality of radially spaced outwardly protruding edges 975 (shown best in FIG. 9) that slideably engage with corresponding grooves 982a of insertion hole 982 of a screw head 984 (shown best in FIG. 10) of the locking feature 950.

With reference to FIG. 10, at least two flexible legs 986 extend downwardly from the screw head 984. Upon insertion of the blade 974b into the insertion hole 982 a user applies downward pressure to flex the legs 986 such that hooks 976 on a distal end of the legs 986 lock into grooves 978 embedded into the primary hub 912. The key 974 can then be removed with the cap 952 remaining locked thereon. In this position the screw head 984 is pushed down into the cap 952 (as shown in FIG. 9). To remove the cap 952, the blade 974b is reentered and twisted either clockwise or counter-clockwise to rotate and release the hooks 976 causing the screw head 984 to pop up and out and align with the screw head 984 with the cap 952 (as shown in FIG. 10). The cap 952 can then be removed exposing the openings 912a, 912b, 912c of the primary hub 912.

While the subject invention has been shown and described with reference to preferred embodiments, those skilled in the art will readily appreciate that various changes and/or modifications may be made thereto without departing from the spirit and scope of the subject invention as defined by the appended claims.

What is claimed is:

1. A catheter assembly comprising:
a) a primary hub having opposed proximal and distal end portions;
b) at least two secondary hubs operatively connected to the proximal end portion of the primary hub, each secondary hub including an elongated body having opposed proximal and distal ends, the proximal end of each secondary hub body adapted and configured for connection with a removable side port.

2. A catheter assembly as recited in claim 1, wherein a multi-lumen catheter body extends from the distal end portion of the primary hub, and includes at least two lumens.

3. A catheter assembly as recited in claim 2, wherein the primary hub is adapted and configured to facilitate communication between the secondary hubs and the multi-lumen catheter.

4. A catheter assembly as recited in claim 2, wherein at least three secondary hubs are operatively connected to the proximal end portion of the primary hub.

5. A catheter assembly as recited in claim 1, wherein a tube extends between the distal end of each secondary hub and the proximal end portion of the primary hub.

6. A catheter assembly as recited in claim 1, wherein an axial passage extends through each secondary hub between the proximal and distal ends thereof, and a hemostatic seal is arranged within the axial passage of each secondary hub.

7. A catheter assembly as recited in claim 1, wherein the proximal end of each secondary hub body includes a leur-type connection for engagement with a fitting associated with a removable side port.

8. A catheter assembly as recited in claim 1, wherein each secondary hub is color matched with a corresponding removable side port.

9. A catheter assembly as recited in claim 1, wherein the primary hub includes suturing structures for securing the primary hub to a patient's body.

10. A catheter assembly as recited in claim 1, further comprising a cap adapted and configured to plug exposed openings of the primary hub when the secondary hubs are removed.

11. A catheter assembly as recited in claim 10, further comprising a locking system utilizing a key configured selectively to lock the cap to the primary hub.

12. A catheter assembly as recited in claim 1, further comprising a fixation system configured to secure the primary hub to a patient's body and prevent movement of the primary hub when not in use.

13. The catheter assembly as recited in claim 12, wherein the fixation system includes a bottom plate having at least two locking posts and a upper plate having holes for receiving the locking posts therein and wherein the primary hub is positioned fixed between the bottom plate and upper plate.

14. A catheter assembly comprising:
a) a primary hub having opposed proximal and distal end portions;
b) a plurality of secondary hubs operatively connected to the proximal end portion of the primary hub, each secondary hub including an elongated body having opposed proximal and distal ends;
c) a multi-lumen catheter body extending longitudinally from the distal end portion of the primary hub;
d) a plurality of removable side ports each adapted and configured for selective connection with the proximal end of a corresponding secondary hub body; and
e) at least one cap adapted and configured to plug exposed openings of the primary hub when the plurality of side ports are removed.

15. A catheter assembly as recited in claim 14, wherein the primary hub is adapted and configured to facilitate communication between the secondary hubs and the multi-lumen catheter.

16. A catheter assembly as recited in claim 14, wherein at least three secondary hubs are operatively connected to the proximal end portion of the primary hub.

17. A catheter assembly as recited in claim 14, wherein at least three removable side ports are operatively associated with the secondary hubs.

18. A catheter assembly as recited in claim 14, further comprising a locking system utilizing a key configured selectively to lock the cap to the primary hub.

19. A catheter assembly as recited in claim 14, further comprising a fixation system configured to secure the primary hub to a patient's body and prevent movement of the primary hub when not in use.

20. The catheter assembly as recited in claim 19, wherein the fixation system includes a bottom plate having at least two locking posts and a upper plate having holes for receiving the locking posts therein and wherein the primary hub is positioned fixed between the bottom plate and upper plate.
